(19) United States
(12) Patent Application Publication
(10) Pub. No.: US 2009/0143695 A1
(43) Pub. Date: Jun. 4, 2009

(54) BRAINWAVE-FACILITATED PRESENTER FEEDBACK MECHANISM

(75) Inventors: Tim R. Mullen, San Pablo, CA (US); Qingfeng Huang, San Jose, CA (US)

Correspondence Address: MARGER JOHNSON & MCCOLLOM/PARC, 210 MORRISON STREET, SUITE 400, PORTLAND, OR, 97204, US

(73) Assignee: PALO ALTO RESEARCH CENTER INCORPORATED, Palo Alto, CA (US)

(21) Appl. No.: 11/947,908

(22) Filed: Nov. 30, 2007

(51) Int. Cl. A61B 5/0484; A61B 5/0476

(52) U.S. Cl. 600/544; 600/300

(57) ABSTRACT

A system includes a presentation subsystem, at least one communications port to receive communications from at least one headset, a processor to process the communications and to produce an aggregated audience response to a presentation on the presentation system, and a status communications module to present the aggregated audience response to a presenter. A computer-controlled method of monitoring interest levels receives at least two signals containing brain wave data for at least one user during a presentation, analyzes the brain wave data to determine a mental state for each user, aggregates the mental states into an aggregate mental state for an audience, and provides the aggregate mental state to a presenter.

### BACKGROUND

Presenters, such as lecturers at academic institutions, presenters at seminars and business meetings, face the task of refining their content and presentation style to maximize interest and ensure understanding and retention of their content. Gauging interest, understanding and focus of the audience presents significant challenges to a presenter. A confusing slide or portion of a presentation can create frustration and lack of interest, much as a boring portion, such as a sequence of boring slides, can cause the audience attention to wander, if not fall asleep altogether.

Another challenge lies in the general reluctance of audiences to provide verbal feedback. Imagine a professor asking, “Do you understand?” and receiving only dead silence. One main obstacle seems to result from the ability of others in the room to identify the feedback provider.

Presenters must rely upon basic visual feedback of watching their audience, or to ask probing questions during presentations. Both of these can detract from the content, as they may serve as distractions. Indeed, listeners may not want to provide feedback as it distracts the listener from the content as well.

With the increase of online instruction provided by academic institutions, as well as continuing education programs and certification programs, some instruction occurs in a prerecorded and asynchronous format, where the presenter and the recipient do not interact at all. Unless recipients provide e-mail or other types of feedback, the presenter has no input as to the efficacy of their material or presentation.

### BRIEF DESCRIPTION OF THE DRAWINGS

FIG. 1 shows an example of an electroencephalogram headset.

FIG. 2 shows an embodiment of an architecture for analyzing brain waves in computing.

FIG. 3 shows an embodiment of a presentation feedback system.

FIG. 4 shows another embodiment of a presentation feedback system.

### DETAILED DESCRIPTION OF THE EMBODIMENTS

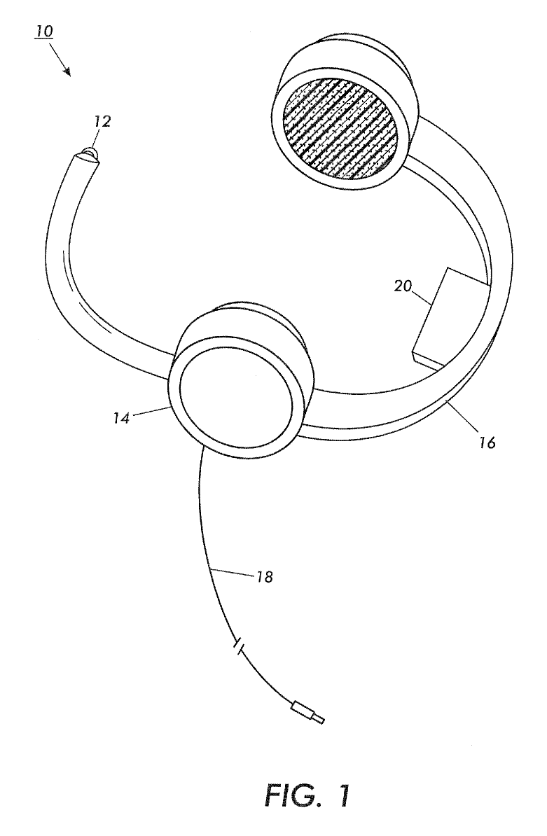

FIG. 1 shows an example of an electroencephalogram (EEG) headset. It must be noted that this is just one example and several other types of headsets or electrode holders may also fall within the scope of the invention as claimed. For example, this headset includes an earphone component that may allow a user to listen to music, but some applications would render earphones undesirable, such as listening to a live presentation.

The headset 10 comprises an electrode 12 attached to one of the ear cups 14 of a pair of earphones 16. Generally, electrodes used in EEGs require a gel between the electrode and the skin. However, recent developments in electrode technologies have resulted in ‘dry’ electrodes that do not require the gel. Daily use by most users would seem to require ‘gelless’ electrodes, or the users will not use the headsets. One example is the dry electrode technology available from Neurosky™ in their ThinkGear™ module.

The headset 10 includes a wire 18 that connects the headset to another device, such as a laptop computer, not shown. The connection would allow transmission of signals both to and from the user. For example, the user may listen to music playing on the laptop, such as from a compact disc (CD) or MP3 music files or podcast, or an online lecture. At the same time, signals from the electrode will transmit to the laptop and/or the network for either real-time or later analysis.

With the improvements in miniaturization and power management, the possibility exists that the headset could become wireless. The user could send and receive signals using wireless technology, such as Bluetooth® technology, similar to cell phone wireless headsets, etc. In the embodiment of FIG. 1, the headset receives power from a battery pack 20. However, using a technology such as USB 2.0, where the device receives power from a USB port on a computer, the battery may become redundant. Alternatively, the battery could also power the wireless transmitter.

In the example above, a laptop or other local computing device received the signals for analysis. This consists of merely one example, as with improvements in wireless technology and miniaturization, the signals could be transmitted to a more remote computer, or even through a wireless access point to a central store. In the example where a local computer receives the signals, the signals could in turn travel to a central store, or other repository for analysis, storage or both. Another possibility involves a processor resident in the headset to also do the analysis. In current circumstances, with current processors, power sources and computing speeds, the analysis will more than likely occur at the local computing device.

The analysis may include many different tasks. For example, the design must select which types of activity to analyze. Much of this will depend upon the nature of the inputs. In the example above, where only one electrode exists, certain representations of the raw data work better than others. For other systems, that may use two or more electrodes, other types of representations may have better accuracy.

The design would then differentiate features of the data, depending upon the nature of the application that will use the data. A ‘feature’ of the data consists of some characteristic, such as a peaks, clusters, phase coherence, etc., that the data analysis will use to determine the meaning of the data.

Once the system has identified the significant features, it then classifies the features. In this particular example, the classification will include a user's mental state, such as boredom, confusion, frustration, interest, etc. One aspect of the mental state could be interest levels in content as the content is presented. Applications can the use the mental state data for various purposes, including as a feedback signal or mechanism.

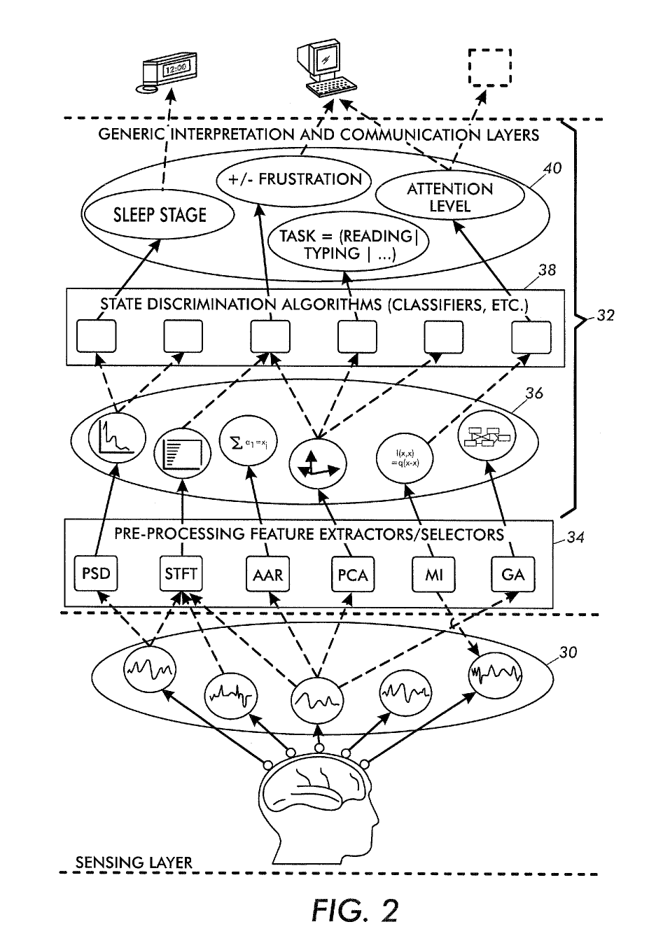

FIG. 2 shows an architecture of a system that selects and classifies the features of brain wave data and then provides it to various applications. The sensing layer 30 may produce a ‘tuplespace’ or associative memory structure. The memory structure consists of tuples, or ordered, such as timestamped, collections of values such as <Electrode 1, [data stream]>. The tuples are then provided to the generic interpretation and communications layer 32.

Within the layer 32, the rectangles such as 34 represent process layers and the ovals such as 36 represent data spaces. The processes, such as 38 can operate asynchronously on the data in the adjacent data spaces. This modularizes the different levels of processing referred to above. Developers working within one layer need not have in-depth expertise regarding implementations of other layers or processes, even within the same layer. Examples of feature extracting methods in 34 may include Principle Component Analysis (PCA), genetic algorithms (GA), Short Time Fourier Transformation (STFT), Adaptive Autoregressive method (AAR), and Power Spectrum Density (PSD).

The ‘top’ of the communication at data space 40 then provides the resulting data to applications that use the data for their own purposes. In this example, the application uses the data to provide feedback to a person giving a presentation, or to a person who is interested in knowing the effectiveness of a presentation, either in real-time or afterwards. It should be noted that the presentation may be of any type of rich media content, such as audio/visual, just audio, just visual, etc.

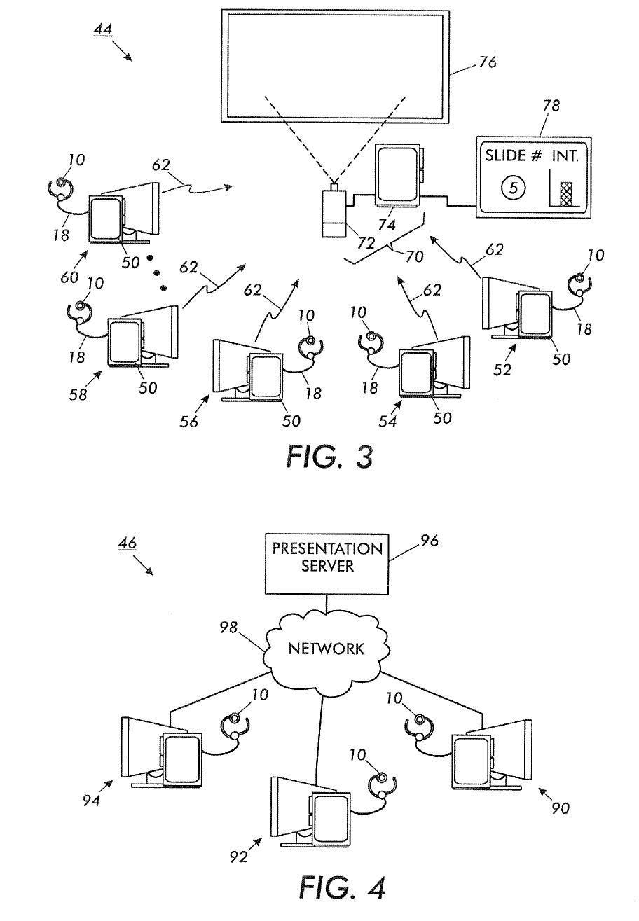

A presentation system 44 including a feedback mechanism is shown in FIG. 3. The presentation system 44 has individual ‘workstations’ 52 in that there are several places for users to view the presentation as it occurs, whether it is a ‘live’ presentation or a video taped or otherwise recorded presentation. Each user/viewer would have a headset such as 10 from FIG. 1, and a laptop or other computing device 50 that receives the signals from the headset. As mentioned previously, each workstation may actually be contained in the wireless headset, but more than likely will be a separate computing device and headset arrangement at this point in time.

Several workstations for several users would exist in the example of FIG. 3, such as workstations 56, 58 and onwards to 60. Each would have some sort of communications like such as 62 to the presentation subsystem 70. Generally, this will be a wireless link between the user computing devices and the aggregator, in this case computer 74.

As embodied here, the presentation subsystem includes a projector 72, a laptop or other computer upon which the presentation resides 74 and a projection screen 76. The signals from the user workstations may arrive at the same computing device 74 in some embodiments, or may be directed to another computing device for analysis.

Similarly, the computing device 74 may include the means for provide the status of the users' attention levels to the presenter, or another device may receive the signals carrying mental state data and then relay them to the device 74. Alternatively, another device may provide the status communications to the presenter.

In operation, as the user views the presentation the headset 10 monitor the brain wave activity through the electrode. The computing device 50 then records the brain wave activity and analyzes it, stores it for later analysis, or transmits it to the computing device 74 for analysis. Regardless of where the analysis occurs, the resulting data represents a user's mental state during the presentation, or at least a portion of the presentation. For example, the presentation may be a typical slide-show presentation during a lecture. A portion of the presentation may be one slide. For a video presentation, a portion of the presentation may be a particular time interval, number of frames or particular video segment.

The brain wave data for each user is analyzed and converted to ‘mental state’ data indicating the user's mental state. As mentioned above, this may involve identifying and selecting features of the data that will then be classified and determined to be the user's mental state. The mental state of the user is then tracked according to the presentation or portion of the presentation.

In one embodiment, a user 's recent learning/activity history maybe used in the analysis of mental reaction to presentation materials, if available. The historical information may help the system to ‘learn’ the user 's response patterns to more accurately portray their mental state with regard to presentation materials.

The system may then gather the mental state of each user and provide an aggregate mental state for the entire audience. References here to ‘aggregate’ or ‘aggregated’ audience response or mental state may include situations in which there is only one user. This information would then be communicated to the presenter, whether the presenter is the current speaker, or just the person who is providing the content. The status communications module may take one of several forms, including a display on the computer 74 upon which the presentation resides. The status display such as 78 may include some means of identifying the portion of the presentation, such as the slide number, and a bar chart or other graph of the indicated interest level.

Many options and variations exist. The analysis of the raw signals from the electrode may occur at the headset, the local computer, the computer upon which the presentation resides or another computer separate from the presentation system. Similarly, the location may vary for the aggregation and preparation of status may vary depending upon a particular system implementation.

The embodiment of the presentation system 44 of FIG. 3 assumes that several users/viewers view the presentation, live or otherwise, at one time. This discussion may refer to this as a ‘real-time’ presentation. The gathering of the brain wave signals occurs at one time for several different users. Another embodiment assumes that several viewers/users view the content or presentation at different times over a longer period of time. FIG. 4 shows an example of this system.

In FIG. 4, several users access the content from their individual workstations such as 90, 92 and 94, each of which has a headset such as 10 from FIG. 1. These individuals may view the content simultaneously for all or a portion of the presentation or they may all view it at separate times. For example, imagine an on-line educational situation where several students in the same class must ‘attend’ a pre-recorded lecture, or view some shared content prepared by their instructor. The workstations could gather their brain waves and transmit across a network 98 them to a central store, such as the server 96, from which they accessed the presentation. As mentioned above, a different server or other device could gather, analyze and store the results of the mental state monitoring.

The system may provide the presenter the ability to set a number of samples, or a length of time, for which it will gather information before providing a feedback result. If there are 200 students in the class, the provider may decide to receive feedback after the first 100 samples come in. Alternatively, the provider may decide to receive feedback after the first week of the content becoming available on the network.

Storing the results could also take many forms. Generally, the overall mental state during the presentation or portions of the presentation would provide feedback to the presenter or creator of the content. However, finer granularity may be desired, such as monitoring of individual attention levels.

Aggregation of mental state data may run the risk of one viewer ‘skewing’ the data by having an unusually high or low interest level, depending upon the number of samples taken. Allowing the users to remain anonymous would have the benefit of ensuring ‘true’ reactions, rather than users worried about appearing bored and giving false data.

In addition to storing more granular levels of data, various levels of synchronization may be stored. The interest levels for each slide could be recorded and synchronized with the mental state/interest level data, or each presentation, each segment of a presentation, etc. Allowing presenters to manipulate and chose the levels of granularity they desire with regard to their feedback provides an added feature to the system.

In addition to the educational environment for either on-line or ‘live’ presentations, the brain wave monitoring and aggregation of mental states could have uses elsewhere. Online content, such as advertisements, web pages, photographs, stories, blogs, etc., could receive evaluations and input from test or focus groups. Real-time, ‘live’ focus groups could view videos, TV advertisements, movie trailers, etc., and provide a different level of feedback to the marketing companies. Any environment where a content provider would like anonymous, ‘automatic’ feedback as to mental states and interest levels caused by their content would find implementations of this invention useful.

In this manner, a system is provided that allows presenters to receive feedback as to the mental state of their audience.

It will be appreciated that several of the above-disclosed and other features and functions, or alternatives thereof, may be desirably combined into many other different systems or applications. Also that various presently unforeseen or unanticipated alternatives, modifications, variations, or improvements therein may be subsequently made by those skilled in the art which are also intended to be encompassed by the following claims.

1. A system, comprising:
a presentation subsystem to allow a presenter to provide a presentation to an audience;
at least one communications port to receive communications from at least one headset worn by a at least one member of the audience;
a processor to process the communications and to produce an aggregated audience response indicating audience interest level in response to [a] the presentation on the presentation system; and
a status communications module to present the aggregated audience response to a presenter at least partially during the presentation.

2. The system of claim 1, wherein the subsystem further comprises a computer having a display, the computer linked to a projector and the status display is displayed on the display.

3. The system of claim 2, wherein the projector further comprises a video projector.

4. The system of claim 1, wherein the processor synchronizes the aggregated audience response to portions of the presentation.

5. The system of claim 1, wherein the portions of the presentation further comprise at least one selected from the group comprised of: slides, video frames, still photographs, audio, and pages of text.

6. The system of claim 1, wherein the processor processes the communications in parallel.

7. The system of claim 1, wherein the status display presents the aggregated audience response associated with a portion of the presentation during a time period in which the portion is being presented at least in part.

8. The system of claim 1, wherein the system further comprises a memory to store statistics associated with the presentation.

9. The system of claim 8, wherein the memory is further to store statistics and the presentation in a synchronized format.

10. The system of claim 1, wherein the communications port further comprises a network port for receiving communications from headsets across a network.

11. The system of claim 1, wherein the communications port further comprises a wireless port for receiving communication from one of either the wireless headsets directly, or through a user computer in communication with the wireless headsets.

12.-17. (canceled)

18. A system, comprising:
a presentation system;
a communications port for receiving transmissions of user brain activity from a plurality of sources during a presentation occurring on the presentation system;
a processor to extract data from the transmissions of user brain activity and to classify the data as mental states of the users to determine a level of interest in response to the presentation; and
a status display to provide a presenter with an aggregate audience response indicating the level of interest in response to the presentation based upon the mental states of the users.

19. The system of claim 18, the system further comprising a memory to store the data and the presentation.

20. The system of claim 18, the communications port to receive transmissions across a network from the plurality of sources.
